(19) United States
(12) Patent Application Publication
(10) Pub. No.: US 2024/0017093 A1
(43) Pub. Date: Jan. 18, 2024

(54) DYNAMIC INTENSITY MODULATION METHOD AND APPARATUS BASED ON ORTHOGONAL DOUBLE-LAYER GRATING ROTARY SWEEPING

(71) Applicant: SUZHOU LINATECH MEDICAL SCIENCE AND TECHNOLOGY CO., LTD., Suzhou, Jiangsu (CN)

(72) Inventors: Yunfei XIANG, Suzhou, Jiangsu (CN); Yao JU, Suzhou, Jiangsu (CN); Jonathan Yi YAO, Suzhou, Jiangsu (CN)

(21) Appl. No.: 18/021,498

(22) PCT Filed: Nov. 26, 2020
(86) PCT No.: PCT/CN2020/131593

§ 371 Date: Feb. 15, 2023

(30) Foreign Application Priority Data
Sep. 8, 2020 (CN) 202010931575.0

(51) Int. Cl. A61N 5/10

(52) U.S. Cl. CPC A61N 5/103; A61N 5/1001; A61N 5/1045; A61N 2005/1094

(57) ABSTRACT

The invention disclosed a dynamic intensity modulation method and device based on orthogonal double-layer grating rotation sweep. The method comprises: 1) obtain the fluence intensity distribution of each beam through the radiotherapy planning system; 2) the beam field is preliminarily divided into four quadrants, which is surrounded by four groups of leaves from the top, bottom, left and right, each quadrant corresponds to the beam intensity distribution in a region within the beam field range, and corresponds to a pair of mutually orthogonal leaves; 3) for the beam intensity distribution in any quadrant, two groups of orthogonal leaves are used for segmentation; 4) synchronize the monitor unit MU of each quadrant; 5) obtain the motion trajectory of active leaves in each quadrant, the motion trajectory of passive leaf in each quadrant and the overall monitor unit MU by calculation. The invention avoids leaf end transmission problem between the closed leaves and reduces the leakage of non-target position, greatly improves the segmentation efficiency, reduces the number of MU required for the plan. It can support two-dimensional dynamic tracking of moving target and lay the foundation for the subsequent treatment of moving target.

### TECHNICAL FIELD

The invention belongs to the field of medical equipment of accelerator radiotherapy, in particular to a dynamic intensity modulation method and device based on orthogonal double-layer grating rotation sweep.

### BACKGROUND

In order to protect healthy tissues from damage, multi-leaf collimator (MLC) is generally used to adjust the irradiation range and intensity of the beam to achieve radiation therapy with adjustable beam intensity. It is called intensity modulated radiotherapy (IMRT).

Multi-leaf collimator (MLC) is initially used in classical conformal radiotherapy to replace the block in conventional radiotherapy and form the desired field shape. MLC consists of two sets of closely packed leaves. Each leaf is made of tungsten alloy, in long strips, and followed by a small motor. Compared with the field block, MLC conformal has significant advantages: it can shorten the treatment time, shorten the time interval between simulated positioning and treatment, and greatly improve the efficiency of radiotherapy; it has stronger attenuation ability to radiation than the block; it is easy and safe to operate without moving heavy blocks; it can be reused; it will not produce harmful gas or dust; it can flexibly respond to changes in target areas and correct errors.

The orthogonal double-layer collimator contains two layers of MLC, which are perpendicular to each other. The corresponding upper leaves and lower leaves cooperate with each other at the edge of the target to achieve the consistency of the shape of MLC and the target boundary and improve the conformity of the field and the target. Because the leaves of at least two layers of leaf collimator device are perpendicular to each other, windows of the same shape can be adjusted according to requirements to block the leakage rays between the leaves, the radiation leakage is greatly reduced, and the penumbra area is effectively reduced, so that the treatment can be accurately located, providing conditions for less fraction and large dose treatment, and the superimposed leaves. The through-leaf collimator radiation is attenuated to a safe range, which improves the efficiency of the equipment and reduces the medical cost and the burden of patients. At the same time, because the upper leaves and lower leaves are perpendicular to each other, they can move in two directions perpendicular to each other.

The current algorithm for MLC dynamic segmentation is mainly the sliding window dynamic scanning segmentation technology. For the combination of the upper and lower layers or multi-layers crossing each other, the sliding window dynamic scanning segmentation technology cannot take into account the leaf movement of the two or multi-layer collimators.

The disadvantages of the existing technology are as follows:
First, the dynamic sliding window scanning segmentation technology makes the collimator move in one direction. Because there is always a gap between the paired closed leaves, there is about 20%-30% of leaf end surface transmission, which cannot be accurately segmented for the complex targets such as concave and ring, resulting in an overall high dose outside the target, a high dose to the organs at risk and a low conformity index of the planning target. The effect of the plan could not meet the requirements;
Second, the efficiency of dynamic sliding-window scanning segmentation technology is sometimes affected by the shape of the target area, and additional head rotation is needed, which has certain requirements for machine tool design;
Third, 2D motion tracking of the moving target is not supported.

### SUMMARY

In order to solve the above technical problems, the invention proposes a dynamic intensity modulation method and device based on orthogonal double-layer grating rotation sweep.

In order to achieve the above purposes, the technical scheme of the invention is as follows:

On the one hand, the invention discloses a dynamic intensity modulation method based on orthogonal double-layer grating rotation sweep, in particular comprising:
1) obtain the fluence intensity distribution of each beam through the radiotherapy planning system;
2) the beam field is preliminarily divided into four quadrants, which is surrounded by four groups of leaves from the top, bottom, left and right, each quadrant corresponds to the beam intensity distribution in a region within the beam field range, and corresponds to a pair of mutually orthogonal leaves;
3) for the beam intensity distribution in any quadrant, two groups of orthogonal leaves are used for segmentation, one group of leaves is active, and the other group is passive, the active leaves move to the center of the beam field along the leaf motion direction, and the passive leaves move out of the beam field along the leaf motion direction;
4) synchronize the monitor unit MU of each quadrant;
5) obtain the motion trajectory of active leaves in each quadrant, the motion trajectory of passive leaves in each quadrant and the overall monitor unit MU by calculation.

On the basis of the above technical scheme, the following improvements can be made:

As a preferred option, the following is also included prior to step 2:
in the isocenter plane, align the intensity map grid obtained from the treatment planning system with the leaf width.

As a preferred option, the following is also included prior to step 2:
in the isocenter plane, align the intensity map grid obtained from the treatment planning system with the leaf width by interpolation method.

As a preferred option, in step 2, the preliminary division of quadrants is divided equally according to the number of leaves in the beam field or according to the complexity of the field intensity map;
the complexity of the field intensity map is defined as the intensity changes in the isocenter plane, or quantified as the accumulation of intensity values along the X-axis or Y-axis. As a preferred option, in step 3, active or passive leaves in adjacent quadrants are not adjacent to each other.

As a preferred option, step 3 comprises the following:
A1) determine the initial position of the leaf, the active leaves are at the edge of the field and the passive leaves are at the junction of the quadrants;
A2) solve the leaf motion trajectory, take the optimized field intensity map of the treatment planning system as the optimization objective, use the multi-segment segmented linear function to fit the local surface, carry out the optimization solution to make the intensity map of the orthogonal leaf motion trajectory meet the requirements, and obtain the ray fluence function f₁(x,y) of the active leaves in each quadrant, the ray shielding function g₂(x,y) of the passive leaves in each quadrant, and the monitor unit MU_{Quad} in each quadrant.

As a preferred option, step 4 comprises the following:
B1) initialize the leaves at quadrant boundary position with serial numbers as Qₓ₁₀, Qₓ₂₀, Q_{y0}, and rank the monitor units in each quadrant from large to small as MUₘₐₓ>MU_{sd}>MUₜₕ>MUₘᵢₙ, if MUₘₐₓ−MUₘᵢₙ<ΔMU, jump out of the subsequent step, wherein ΔMU is the maximum monitor unit difference allowed in the quadrant;
B2) find the quadrant of MUₘᵢₙ and MUₘₐₓ;
   if MUₘₐₓ and MUₘᵢₙ respectively in the first quadrant and the second quadrant, adjust the leaves with serial number Qₓ₁ to reduce MUₘₐₓ and to increase MUₘᵢₙ;
   if MUₘₐₓ and MUₘᵢₙ respectively in the third quadrant and the fourth quadrant, adjust the leaves with serial number Qₓ₂ to reduce MUₘₐₓ and to increase MUₘᵢₙ;
   if MUₘₐₓ and MUₘᵢₙ respectively in the first quadrant and the fourth quadrant, or MUₘₐₓ and MUₘᵢₙ, respectively, in the second quadrant and the third quadrant, then adjust the leaves with serial number Q_{y} to reduce MUₘₐₓ and to increase MUₘᵢₙ;
   if MUₘₐₓ and MUₘᵢₙ on the quadrant of the diagonal respectively, and MU_{sd} with MUₘᵢₙ is located in the same line, then adjust the leaves with serial number Q_{y} to reduce MUₘₐₓ and to increase MUₘᵢₙ;
   if MUₘₐₓ and MUₘᵢₙ on the quadrant of the diagonal respectively, and MU_{sd} with MUₘᵢₙ, that are in the same column, adjust the leaves with serial number Qₓ₁ and the leaves with serial number Qₓ₂ to reduce MUₘₐₓ and to increase MUₘᵢₙ;
B3) adjust the leaves with serial numbers Qₓ₁, Qₓ₂ and Q_{y}, and perform quadrant segmentation calculation again through step 3 to obtain the ray fluence function f₁(x,y) of active leaf in each quadrant, the ray shielding function g₂(x,y) of passive leaves in each quadrant and the monitor unit MU_{Quad} in each quadrant, and return to step B1.

As a preferred option, step 5 comprises the following:
record the ray fluence function f₁(x,y) of the active leaves, the ray shielding function g₂(x,y) of the passive leaves, and the maximum monitor unit MUₘₐₓ of each quadrant calculated by the last orthogonal segmentation, the ray fluence function f₁(x,y) and the ray shielding function g₂(x,y) are the motion trajectories of active and passive leaves by unit conversion, and MUₘₐₓ is the overall monitor unit MU.

On the other hand, the invention also disclose a dynamic intensity modulation device based on orthogonal double-layer grating rotation sweep, including a computer and a program implemented with the computer for performing the dynamic intensity modulation method based on orthogonal double-layer grating rotation sweep for any of the above options.

A dynamic intensity modulation method and device based on orthogonal double-layer grating rotation sweep have the following beneficial effects:
First, the dynamic intensity modulated problem of orthogonal double-layer collimator is solved. The dynamic segmentation of arbitrary shape target area (concave target area, ring target area, etc.) and multi-target area are completed by the coordinated movement of the upper and lower orthogonal leaves. The dynamic segmentation of the upper and lower orthogonal double-layer collimator from two directions is realized to avoid the end transmission problem between the closed leaves and reduce the transmission of non-target areas. Improve the effect of the plan and reduce the difficulty of the plan production.
Second, at the same time, the segmentation efficiency is greatly improved, the monitor unit MU required for the plan is reduced, the leaf movement stroke is reduced, and the machine energy consumption and loss are reduced.
Third, it can support two-dimensional dynamic tracking of the moving target, laying a foundation for the subsequent treatment of moving target.

### BRIEF DESCRIPTION OF THE DRAWINGS

In order to more clearly illustrate the technical solutions of embodiments of the present invention, a brief introduction will be made to the figures required to be used in the embodiments below. It should be understood that the figures below show only certain embodiments of the present invention and therefore should not be regarded as a limitation of the scope. For those of ordinary skill in the art, without creative labor, other relevant supplementary figures can also be obtained based on these supplementary figures.

FIG. 1 shows a three-dimensional view of the fluence intensity distribution of the target area of nasopharyngeal carcinoma provided by an embodiment of the present invention.

FIG. 2 (a) a schematic diagram of the position of the orthogonal double-layer collimator, the ray source and the isocenter plane provided in an embodiment of the invention;

FIG. 2 (b) shows the position distribution diagram of the orthogonal double-layer collimator provided by the embodiment of the invention in the field coordinate system.

FIG. 3 shows a view of the relationship between an orthogonal double-layer collimator leaf and an optimized intensity map grid provided by an embodiment of the invention.

FIG. 4 provides a diagram of quadrant division for an embodiment of the invention.

FIG. 5 shows a schematic diagram of an initial position situation of a leaf in the first quadrant provided by an embodiment of the invention.

FIG. 6 shows a schematic diagram of the relationship between leaf motion and fluence in the orthogonal leaf overlap area provided by an embodiment of the invention.

FIG. 7 shows a schematic diagram of quadrant division and leaf allocation provided by an embodiment of the invention.

FIGS. 8(a)-8(f) show a schematic diagram of the synchronization of the monitor units in each quadrant provided by an embodiment of the invention.

FIG. 9 shows a schematic diagram of the relationship between the ray fluence projection of a nasopharyngeal carcinoma case and the initial position of leaves in each quadrant provided by an embodiment of the invention.

FIG. 10 shows the fluence intensity distribution map provided by the embodiment of the present invention using the dynamic intensity modulation method based on orthogonal double-layer grating rotation sweep.

FIG. 11 shows a flowchart of a dynamic intensity modulation method and device based on orthogonal double-layer grating rotation sweep provided by an embodiment of the invention.

Where: 11—front side of upper collimator, 12—back side of upper collimator, 13—left side of lower collimator, 14—right side of lower collimator, A—overlap area;

2—active leaf, 3—passive r leaf.

### DETAILED DESCRIPTION OF THE EMBODIMENTS

Preferred embodiments of the invention are described in detail below in conjunction with the accompanying figures.

The technical solutions in the embodiments of the present invention will be clearly and completely described below in conjunction with the figures attached to the embodiments of the present invention, and it is evident that the described embodiments are only some embodiments of the present invention, but not all embodiments. Based on the embodiments of the present invention, all other embodiments obtained by a person of ordinary skill in the art without creative labor are within the sc.

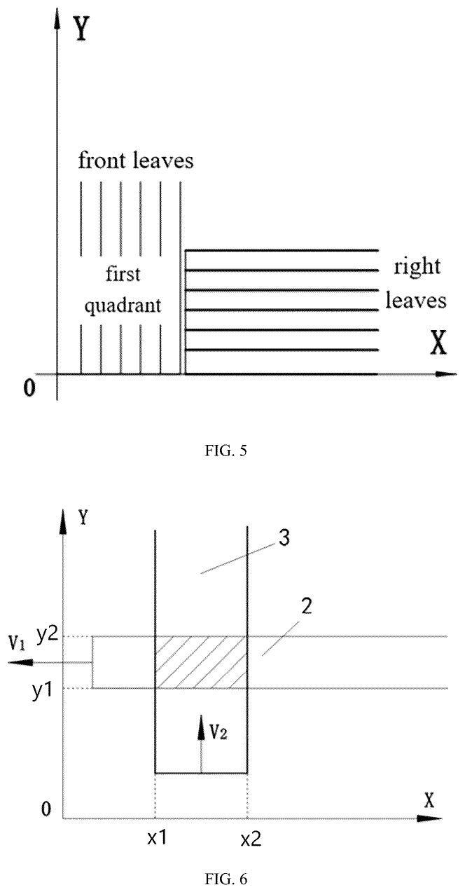

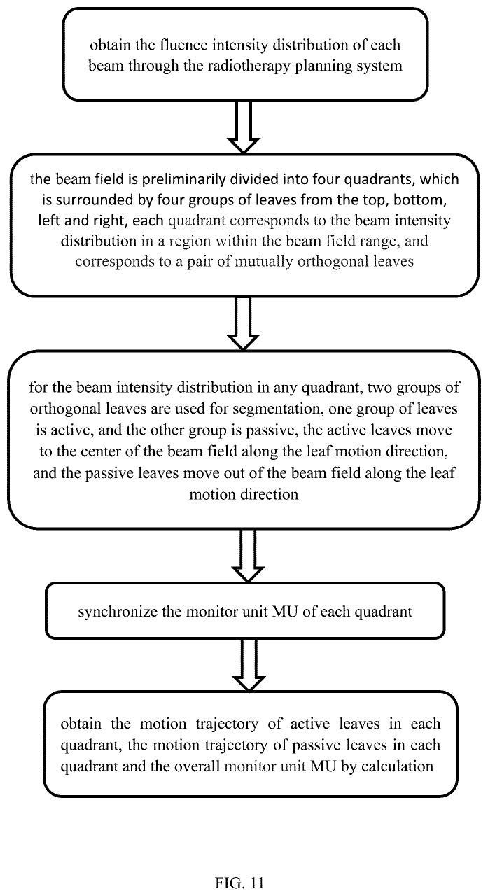

As shown in FIG. 11, an embodiment of the invention discloses a dynamic intensity modulation method based on orthogonal double-layer grating rotation sweep, in particular comprising:
1) obtain the fluence intensity distribution of each beam through the radiotherapy planning system, in the field range of the isocenter plane, the fluence intensity value is represented as a curved surface, and the fluence intensity value at any point on the field plane is denoted as I(x,y);
2) as shown in FIG. 4, the beam field is preliminarily divided into four quadrants, which is surrounded by four groups of leaves from the top, bottom, left and right, each quadrant corresponds to the beam intensity distribution in a region within the beam field range, and corresponds to a pair of mutually orthogonal leaves;
3) for the beam intensity distribution in any quadrant, two groups of orthogonal leaves are used for segmentation, one group of leaves is active and the other group is passive, the active leaves move to the center of the beam field along the leaf motion direction, and the passive leaves move out of the beam field along the leaf motion direction;
4) synchronize the monitor unit MU of each quadrant;
5) obtain the motion trajectory of active leaves in each quadrant, the motion trajectory of passive leaves in each quadrant and the overall monitor unit MU by calculation. In order to further optimize the embodiments of the invention, in some other embodiments the rest of the characteristic techniques are the same, except that the following is also included before step 2:
in the isocenter plane, align the intensity map grid obtained from the treatment planning system with the leaf width.

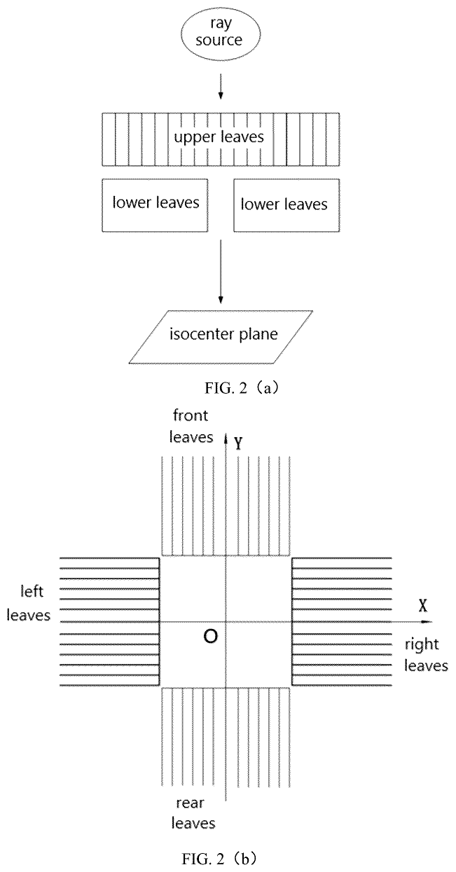

As shown in FIG. 2(a), the orthogonal double-layer collimator is used for dynamic intensity modulation. The collimator is installed between the ray source and the isocenter plane, and the ray is projected on the coordinate system S-XY of the isocenter plane through the upper and lower layers of collimator. The upper leaves and lower leaves are located in four directions respectively, as shown in FIG. 2 (b), wherein the upper leaves are at the front and rear ends layer and lower leaves are at the left and right ends.

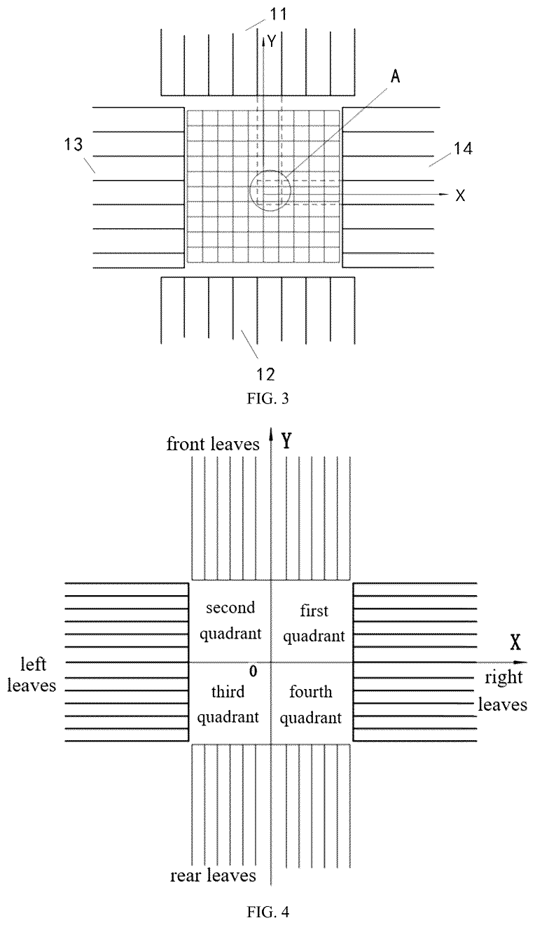

As shown in FIG. 3, generally, in the isocenter plane, the intensity map grid and leaf width obtained by the treatment planning system are not necessarily aligned, and the intensity map corresponding to the grid of the leaf width of the orthogonal double-layer collimator can be obtained by but not limited to the interpolation method. In FIG. 3, 11 represents the front side of the upper collimator, 12 represents the back side of the upper collimator, 13 represents the left side of the lower collimator, and 14 represents the right side of the lower collimator.

The general leaf width is around 10 mm, and the leaf width of different manufacturers is slightly different. In order to get more accurate results, the grid formed by a pair of mutually orthogonal leaves is defined as the overlap area A, and then the leaf width is divided equally into N interpolation points to obtain a more fine-grained numerical surface, i.e., each intersection region has N×N intensity value points, and the more fine-grained flux intensity matrix is noted as Iₒₚₜ.

In order to further optimize the implementation of the present invention, in some other implementations, the remaining features are technically identical, with the difference that in step 2, the preliminary division of quadrants is divided equally according to the number of leaves in the beam field or according to the complexity of the field intensity map.

The complexity of the field intensity map is defined as the intensity changes in the isocenter plane, or quantified as the accumulation of intensity values along the X-axis or Y-axis. To further optimize the implementation of the present invention, in some other embodiments, the remaining features are technically identical, with the difference that in step 3, the active leaves or passive leaves in adjacent quadrants are not adjacent to each other.

In order to further optimize the implementation effect of the present invention, in some other embodiments, the other characteristic techniques are the same, the difference is that step 3 is as follows:
A1) determine the initial position of the leaf, the active leaves are at the edge of the field and the passive leaves are at the junction of the quadrants.

As mentioned above, each quadrant contains a group of horizontal and a group of vertical collimator leaves. One group of leaves is defined as active leaves, and the other group of leaves is defined as passive leaves. The active leaves move to the center of the beam field along the leaf motion direction, and the passive leaves move outward from the beam field along the leaf motion direction. The active leaves or passive leaves in adjacent quadrants are not adjacent to each other. Therefore, the initial position can be determined as follows: the active leaves are at the edge of the field, and the passive leaves are at the quadrant junction position.

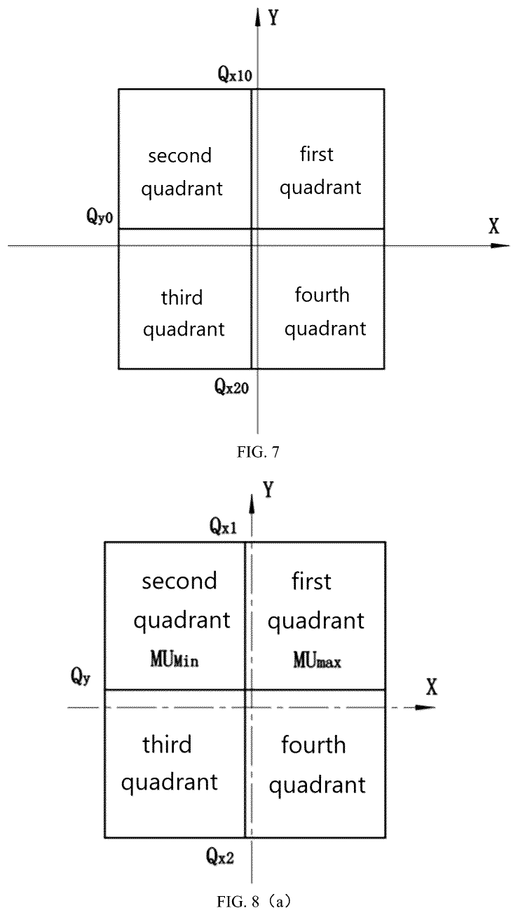

As shown in FIG. 5, is an initial position situation of the leaves in the first quadrant, with the right leaf as a group of active leaves and the left leaf as a group of passive leaves. In general, if the intensity value of rays in the field near the edge is zero, the initial position of the active leaves can be moved into the field to reduce the radiation leakage.
A2) solve the leaf motion trajectory, take the optimized field intensity map of the treatment planning system as the optimization objective, use the multi-segment segmented linear function to fit the local surface, carry out the optimization solution to make the intensity map of the orthogonal leaf motion trajectory meet the requirements, and obtain the ray fluence function f₁(x,y) of the active leaves in each quadrant, the ray shielding function g₂(x,y) of the passive leaves in each quadrant, and the monitor unit MU_{Quad} in each quadrant.

As mentioned above, the starting position of the leaf is determined, and the leaf motion trajectory is solved in step A2, so that the ray intensity through the field is consistent with the results optimized by the treatment planning system .

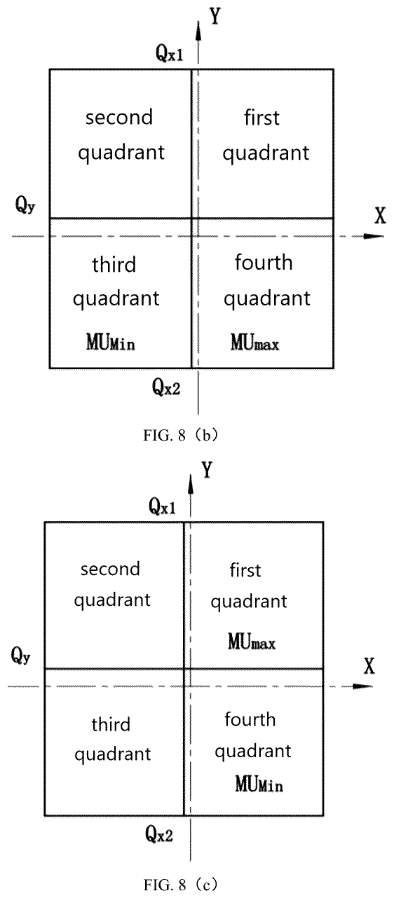

Known that, the fluence intensity of is optimized for the Iₒₚₜ(x,y), wherein (x, y) is the location of the isocenter coordinate system. As shown in FIG. 6, the active leaf 2 moves with speed v₁ in the horizontal direction, and the passive leaf 3 moves with speed v₂ in the vertical direction. The fluence intensity distribution in this overlap area is:

I_{deli}(x,y)=f₁(x,y)−g₂(x,y):

Among them, the I_{deli}(x,y) as the leaf motion segmentation of fluence intensity;
f₁(x,y) is the intensity of ray passing at the point (x,y) without considering the occlusion of the passive leaves;
g₂(x,y) is the ray intensity occluded by the passive leaves at the point (x,y) position.

Assume that the velocity function of the active leaves movement is v1(x), v₁(x) changes along the X-axis. The velocity function of the passive leaves movement is v₂(y), and v₂(y) changes along the Y-axis.

Then the fluence intensity at any point P(x′,y′) in the overlap area is

$$\{ \begin{matrix}{f}_{1} ( {x}^{′} , {y}^{′} ) = {R}_{d o s e} \cdot {\int }_{{x}_{2}}^{{x}^{′}} \frac{1}{{v}_{1} ( x )} d x + {f}_{1} ( {x}_{2} , {y}^{′} ) \\ {g}_{2} ( {x}^{′} , {y}^{′} ) = {R}_{d o s e} \cdot {\int }_{{y}_{1}}^{{y}^{′}} \frac{1}{{v}_{2} ( y )} d y + {g}_{2} ( {x}^{′} , {y}_{1} ) \\ {I}_{deli} ( {x}^{′} , {y}^{′} ) = {f}_{1} ( {x}^{′} , {y}^{′} ) - {g}_{2} ( {x}^{′} , {y}^{′} ) \\ \end{matrix} ;$$

Where, R_{dose} is the dose rate of the accelerator.

Given that the ray target flux intensity value in the overlap area is Iₒₚₜ, the problem of solving the leaf path can be transformed into an optimization problem of solving the leaf velocity function, so that the fluence intensity value I_{deli} segmented by orthogonal leaf motion is consistent with Iₒₚₜ. The mathematical model of the optimization problem is as follows:

min ∫ₓ₂^{x1}∫_{y1}^{y2}(I_{deli}(x,y)−Iₒₚₜ(x,y))²dxdy

st. V₁ₘᵢₙ<v₁(x)<V₁ₘₐₓ;

V₂ₘᵢₙ<v₂(y)<V₂ₘₐₓ

The leaf velocity functions v₁(x) and v₂(y), the ray fluence function f₁(x,y) of the active leaves and the ray shielding function g₂(x,y) of the passive leaves are obtained by optimizing the solution.

Where, the maximum number of monitor units in this quadrant is MU_{Quad} is:

MU_{Quad}=max(f₁(x,y)).

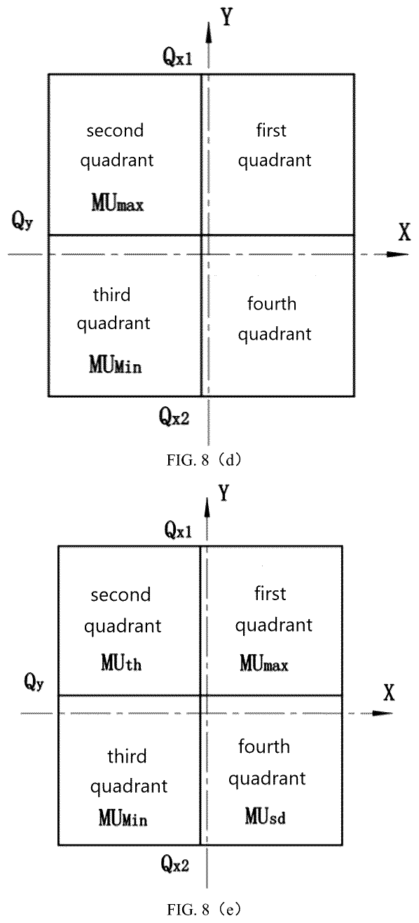

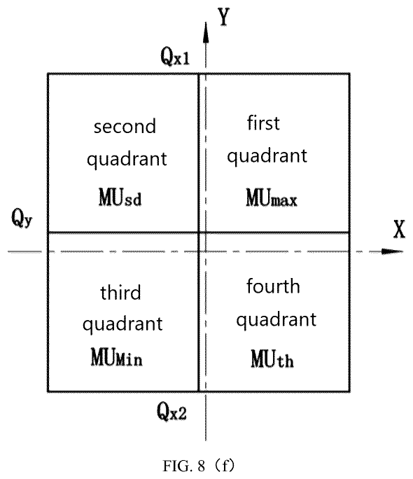

In order to further optimize the implementation effect of the invention, on the basis of the above implementation, in order to ensure that the overall MU of the machine is the smallest, the MU of each quadrant is as consistent as possible, and the allocation of the quadrants needs to be adjusted. Step 4 is as follows:
B1) As shown in FIG. 7, the MU of the first, second, third and fourth quadrants after orthogonal segmentation are MU_{Quad1}, MU_{Quad2}, MU_{Quad3} and MU_{Quad4}; The number of MU in each quadrant is ranked from large to small as MUₘₐₓ>MU_{sd}>MUₜₕ>MUₘᵢₙ, if MUₘₐₓ−MUₘᵢₙ<ΔMU, the subsequent step would be exited, where ΔMU is the maximum allowed difference of MU in each quadrant;
B2) find the quadrant of MUₘᵢₙ and MUₘₐₓ;
as shown in FIG. 8 (a), if MUₘₐₓ and MUₘᵢₙ respectively in the first quadrant and the second quadrant, adjust the leaves with serial number Qₓ₁ to reduce MUₘₐₓ and to increase MUₘᵢₙ;
as shown in FIG. 8 (b), if MUₘₐₓ and MUₘᵢₙ respectively in the third quadrant and the fourth quadrant, adjust the leaves with serial number Qₓ₂ to reduce MUₘₐₓ and to increase MUₘᵢₙ;
as shown in FIG. 8 (c) and FIG. 8 (d), if MUₘₐₓ and MUₘᵢₙ respectively in the first quadrant and the fourth quadrant, or MUₘₐₓ and MUₘᵢₙ, respectively, in the second quadrant and the third quadrant, then adjust the leaves with serial number Q_{y} to reduce MUₘₐₓ and to increase MUₘᵢₙ; as shown in FIG. 8 (e), if MUₘₐₓ and MUₘᵢₙ on the quadrant of the diagonal respectively, and MU_{sd} with MUₘᵢₙ is located in the same line, then adjust the leaves with serial number Q_{y} to reduce MUₘₐₓ and to increase MUₘᵢₙ;
as shown in FIG. 8 (f), if MUₘₐₓ and MUₘᵢₙ on the quadrant of the diagonal respectively, and MU_{sd} with MUₘᵢₙ, that are in the same column, adjust the leaves with serial number Qₓ₁ and the leaves with serial number Qₓ₂ to reduce MUₘₐₓ and to increase MUₘᵢₙ;
B3) adjust the leaves with serial numbers Qₓ₁, Qₓ₂ and Q_{y}, and perform quadrant segmentation calculation again through step 3 to obtain the ray fluence function f₁(x,y) of active leaves in each quadrant, the ray shielding function g₂(x,y) of passive leaves in each quadrant and the monitor unit MU_{Quad} in each quadrant, and return to step B1.

In order to further optimize the implementation effect of the invention, on the basis of the above embodiments, step 5 is:
record the ray fluence function f₁(x,y) of the active leaves, the ray shielding function g₂(x,y) of the passive leaves, and the maximum monitor unit MUₘₐₓ of each quadrant calculated by the last orthogonal segmentation , the ray fluence function f₁(x,y) and the ray shielding function g₂(x,y) are the motion trajectories of active and passive leaves by unit conversion, and MUₘₐₓ is the overall monitor unit MU.

On the other hand, an embodiment of the invention also disclose a dynamic intensity modulation device based on orthogonal double-layer grating rotation sweep, including a computer and a program implemented by a computer for performing a dynamic intensity modulation method based on orthogonal double-layer grating rotation sweep for any of the above schemes.

A dynamic intensity modulation method and device based on orthogonal double-layer grating rotation sweep have the following beneficial effects:
First, the dynamic intensity modulated problem of orthogonal double-layer collimator is solved. The dynamic segmentation of arbitrary shape target area (concave target area, ring target area, etc.) and multi-target area are completed by the coordinated movement of the upper and lower orthogonal leaves. The dynamic segmentation of the upper and lower orthogonal double-layer collimator from two directions is realized to avoid the leaf end transmission problem between the closed leaves and reduce the transmission of non-target areas. Improve the effect of the plan and reduce the difficulty of the plan production.
Second, at the same time, the segmentation efficiency is greatly improved, the monitor unit MU required for the plan is reduced, the leaf movement stroke is reduced, and the machine energy consumption and loss are reduced.
Third, it can support two-dimensional dynamic tracking of the moving target, laying a foundation for the subsequent treatment of moving target.

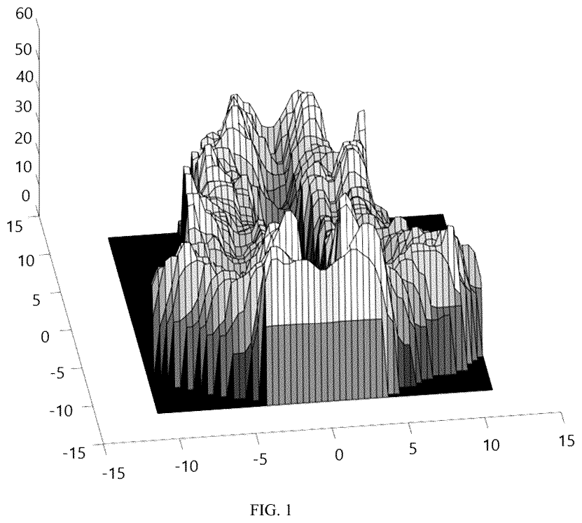

In order to illustrate the specific implementation process of the invention, a case of nasopharyngeal carcinoma is illustrated. The specific process is as follows:
Step 1) obtain the fluence intensity matrix of each beam through the radiotherapy planning system, in the field range of the isocenter plane, it could be expressed as I_{opt(x,y)}. Its three-dimensional view is shown in FIG. 1, and its height direction represents the fluence intensity value.
Step 2) As shown in FIG. 7, the initial quadrants are divided according to the complexity of the fluence intensity matrix of the field, and the fluence distribution of the four quadrants and the serial numbers of the junctional leaves Qₓ₁₀, Qₓ₂₀, and Q_{y0} are obtained.

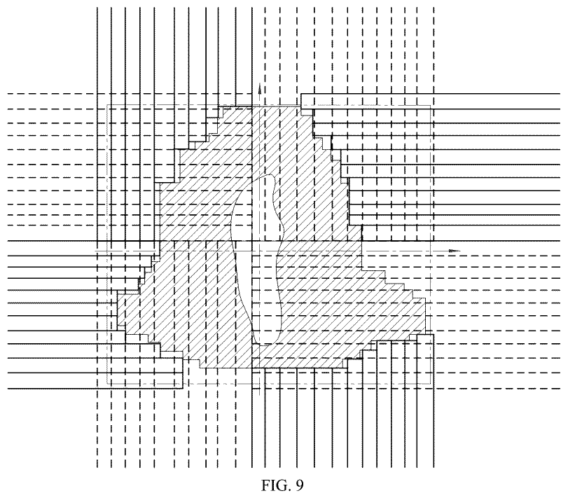

As shown in FIG. 9, the shaded part in the figure is the projection of the field fluence on the isocenter plane, and the thick solid line represents the active leaves, whose initial position is close to the field fluence profile. The thick dashed line represents the passive leaves, whose initial position is at the quadrant division junction.
Step 3) Each quadrant is solved by orthogonal segmentation, as shown in FIG. 6. Assume that the active leaves move with speed v₁ in the horizontal direction and the passive leaves move with speed v₂ in the vertical direction, the fluence intensity distribution in the overlap area is:

I_{deli}(x,y)=f₁(x,y)−g₂(x,y);

Among them, the I_{deli}(x, y) as the leaf motion segmentation of fluence intensity;
f₁(x,y) is the intensity of ray passing at the point (x,y) without considering the occlusion of the passive leaves;
g₂(x,y) is the ray intensity occluded by the passive leaves at the point (x,y) position.

Take any one of overlap areas, and assume that the velocity function of the active leaves movement is v₁(x), v₁(x) changes along the X-axis. The velocity function of the passive leaves movement is v₂(y), and v₂(y) changes along the Y-axis.

Then the fluence intensity at any point P(x′,y′) in the overlap area is:

$$\{ \begin{matrix}{f}_{1} ( {x}^{′} , {y}^{′} ) = {R}_{d o s e} \cdot {\int }_{{x}_{2}}^{{x}^{′}} \frac{1}{{v}_{1} ( x )} d x + {f}_{1} ( {x}_{2} , {y}^{′} ) \\ {g}_{2} ( {x}^{′} , {y}^{′} ) = {R}_{d o s e} \cdot {\int }_{{y}_{1}}^{{y}^{′}} \frac{1}{{v}_{2} ( y )} d y + {g}_{2} ( {x}^{′} , {y}_{1} ) \\ {I}_{deli} ( {x}^{′} , {y}^{′} ) = {f}_{1} ( {x}^{′} , {y}^{′} ) - {g}_{2} ( {x}^{′} , {y}^{′} ) \\ \end{matrix} ;$$

Where, R_{dose} is the dose rate of the accelerator.

Knowing the ray target flux intensity value of Iₒₚₜ in the overlap area, the problem of solving the leaf path is transformed into an optimization problem of solving the leaf velocity function so that the orthogonal leaf motion splitting out the fluence intensity value I_{deli} is consistent with Iₒₚₜ. The mathematical model of the optimization problem is as follows.

min ∫ₓ₂^{x1}∫_{y1}^{y2}(I_{deli}(x,y)−Iₒₚₜ(x,y))²dxdy

st. V₁ₘᵢₙ<v₁(x)<V₁ₘₐₓ

V₂ₘᵢₙ<v₂(y)<V₂ₘₐₓ

The mathematical problem is discretized and solved, and the weighting effect of the segmentation efficiency is considered, which can be converted into the following multi-objective optimization mathematical model.

$$\begin{matrix}min & F = {w}_{1} \cdot {F}_{1} + {w}_{2} \cdot {F}_{2} + {w}_{3} \cdot {F}_{3}\end{matrix}$$

$${F}_{1} = {\sum }_{i = 1}^{N} {\sum }_{j = 1}^{N} {[ ( \frac{1}{{v}_{1} ( {x}_{1} )} + \frac{1}{{v}_{1} ( {x}_{2} )} + … + \frac{1}{{v}_{1} ( {x}_{i} )} ) \cdot Δ x \cdot {R}_{d o s e} + {f}_{1} ( {x}_{1} , y ) - ( \frac{1}{{v}_{2} ( {y}_{1} )} + \frac{1}{{v}_{2} ( {y}_{2} )} + … + \frac{1}{{v}_{2} ( {y}_{j} )} ) \cdot Δ y \cdot {R}_{d o s e} - {g}_{2} ( x , {y}_{1} ) - {I}_{opt} ( {x}_{i} , {y}_{j} ) ]}^{2}$$

$${f}_{1} ( {x}_{i} , y ) = ( \frac{1}{{v}_{1} ( {x}_{1} )} + \frac{1}{{v}_{1} ( {x}_{2} )} + … + \frac{1}{{v}_{1} ( {x}_{i} )} ) \cdot Δ x \cdot {R}_{d o s e} + {f}_{1} ( {x}_{1} , y )$$

$${g}_{2} ( x , {y}_{j} ) = ( \frac{1}{{v}_{2} ( {y}_{1} )} + \frac{1}{{v}_{2} ( {y}_{2} )} + … + \frac{1}{{v}_{2} ( {y}_{j} )} ) \cdot Δ y \cdot {R}_{d o s e} + {g}_{2} ( x , {y}_{1} )$$

$${F}_{2} = ( \frac{1}{{v}_{1} ( {x}_{1} )} + \frac{1}{{v}_{1} ( {x}_{2} )} + … + \frac{1}{{v}_{1} ( {x}_{N} )} ) \cdot Δ x$$

$${F}_{3} = ( \frac{1}{{v}_{2} ( {x}_{1} )} + \frac{1}{{v}_{2} ( {x}_{2} )} + … + \frac{1}{{v}_{3} ( {x}_{N} )} ) \cdot Δ y$$

where [w₁ w₂ w₃] is the objective function weight value, F₁ represents the vector 2-norm value of the difference between the segmented fluence intensity values I_{deli} and Iₒₚₜ; F₂ represents the time accumulated by the active leaves through the overlap area; F₃ represents the time accumulated by the passive leaves through the overlap area. The final solution yields the ray fluence function f₁(x,y) of the active leaves in each quadrant, the ray shielding function g₂(x,y) of the passive leaves in each quadrant, and the monitor unit MU_{Quad} in each quadrant.
Step 4) Synchronize the monitor unit MU of each quadrant and adjust Qₓ₁, Qx₂ and Q_{y}, so that MUₘₐₓ−MUₘᵢₙ<ΔMU.
Step 5) The ray fluence function f₁(x,y) of the active leaves, the ray shielding function g₂(x,y) of the passive leaves and the maximum monitor unit MUₘₐₓ of each quadrant calculated by the last orthogonal segmentation, the ray fluence function f₁(x,y) and the ray shielding function g₂(x,y) are the motion trajectories of active and passive leaves by unit conversion, and MUₘₐₓ is the overall monitor unit MU.

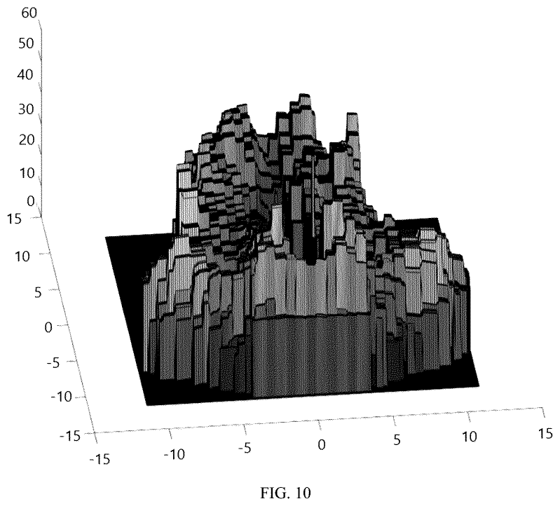

As shown in FIG. 10, for the fluence intensity map of one of the fields obtained by rotating sweep with orthogonal double-layer collimator, the MU required by dynamic intensity modulation with orthogonal double-layer collimator is shown in Table 1 for the selected cases. Compared with the sliding window algorithm with single-layer collimator, the MU is reduced by 16.7% overall.

TABLE 1
MU contrasts required for dynamic intensity modulation using
orthogonal double-layer collimators in selected cases

| | double layer | Single layer | The double |
|---|---|---|---|
| | collimator | collimator | layer MU |
| | rotate sweep | sliding window | reduces the |
| Beam | MU | MU | relative value |
| beam1 | 187 | 259 | −27.88% |
| beam2 | 178 | 212 | −16.04% |
| beam3 | 123 | 151 | −18.69% |
| beam4 | 178 | 178 | 0.20% |
| beam5 | 174 | 227 | −23.39% |
| beam6 | 194 | 238 | −18.37% |
| beam7 | 152 | 195 | −22.19% |
| beam8 | 131 | 124 | 5.33% |
| beam9 | 176 | 208 | −15.21% |
| Total MU | 1493 | 1792 | −16.70% |

The invention provides a dynamic intensity modulation method and device based on orthogonal double-layer grating rotation sweep. The method of quadrant is adopted, and four groups of collimator leaves are assigned to different quadrants. Each quadrant contains a group of horizontal and a group of vertical collimator leaves, among which one group of leaves is active and the other group of leaves is passive. Among them, the active leaves move to the center of the field along the leaf motion direction, and the passive leaves move to the field along the leaf motion direction. The active leaves (passive leaves) in the adjacent quadrants are not adjacent to each other, and the four quadrants are segmented synchronously to form a rotating sweep dynamic intensity modulated mode. It also proposed a method to solve the dynamic intensity modulation of a pair of orthogonal leaves. Take the optimized beam intensity map of the radiotherapy planning system as the optimization objective, the multi-segment linear function is used to fit the local surface, and the optimization solution is carried out to make the intensity map of the orthogonal leaf motion trajectory meet the requirements. At the same time, a four-quadrant synchronization method is proposed to make the movement of the four quadrants synchronized, while reducing the overall MU.

The beneficial results of the present invention compared with the prior art specifically include:
1. Improve the efficiency of dynamic intensity modulation: the quadrant-segmented rotating sweep intensity modulation segmentation method is used to optimize the search of better segment sequence in the two-dimensional space, so that the overall MU is greatly reduced under the same collimator parameters.
2. The dose intensity outside the planning target volume is reduced: the dose outside the planning target volume is greatly reduced by the cross shielding of the upper and lower collimators.
3. Enhance protection of crisis organs: the upper and lower layers of the double-layer collimator are used to better shield and protect the crisis organs and avoid high doses.
4. Improve the conformity of the target: the orthogonal double-layer collimator can conformal the target contour from two directions and improve the conformity of the target.
5. Realize the intensity segmentation of multi-target areas: the combination of four sets of leaves with different orientations can be used to divide up to four quadrants, and the multi-target problems within four can be segmented simultaneously.
6. Two-dimensional dynamic tracking treatment of the moving target: a pair of orthogonal leaves is used to segment the target area, which can realize two-dimensional dynamic tracking treatment of the moving target area.
7. Improve the service life of multi-leaf collimator MLC: By using quadrant intensity modulation, the leaves only travel half of the original position, the motor running time is shortened, the wear of the lead screw is greatly reduced, and the overall life of MLC is significantly improved. At the same time, the design requirements for the length of multi-leaf collimator leaves are also reduced.

The above examples only illustrate the technical conception and characteristics of the invention, which aims to allow ordinary technicians in the art to understand the content of the invention and implement it, and can not limit the scope of protection of the invention. Any equivalent changes or modifications made according to the essence of the invention should be covered by the scope of protection of the invention.

1. Dynamic intensity modulation method based on orthogonal double-layer grating rotation sweep, comprising:
1) obtain the fluence intensity distribution of each beam through the radiotherapy planning system;
2) the beam field is preliminarily divided into four quadrants, which is surrounded by four groups of leaves from the top, bottom, left and right, each quadrant corresponds to the beam intensity distribution in a region within the beam field range, and corresponds to a pair of mutually orthogonal leaves;
3) for the beam intensity distribution in any quadrant, two groups of orthogonal leaves are used for segmentation, one group of leaves is active, and the other group is passive, the active leaves move to the center of the beam field along the leaf motion direction, and the passive leaves move out of the beam field along the leaf motion direction;
4) synchronize the monitor unit MU of each quadrant;
5) obtain the motion trajectory of active leaves in each quadrant, the motion trajectory of passive leaf in each quadrant and the overall monitor unit MU by calculation.

2. The dynamic intensity modulation method based on orthogonal double-layer grating rotation sweep according to claim 1, prior to step 2, the following is also included:
in the isocenter plane, align the intensity map grid obtained from the treatment planning system with the leaf width.

3. The dynamic intensity modulation method based on orthogonal double-layer grating rotation sweep according to claim 2, prior to step 2, the following is also included:
in the isocenter plane, align the intensity map grid obtained from the treatment planning system with the leaf width by interpolation method.

4. The dynamic intensity modulation method based on orthogonal double-layer grating rotation sweep according to claim 1, in step 2, the preliminary division of quadrants is divided equally according to the number of leaves in the beam field or according to the complexity of the field intensity map;
the complexity of the field intensity map is defined as the intensity changes in the isocenter plane, or quantified as the accumulation of intensity values along the X-axis or Y-axis.

5. The dynamic intensity modulation method based on orthogonal double-layer grating rotation sweep according to claim 1, in step 3, the active leaves or passive leaves in adjacent quadrants are not adjacent to each other.

6. The dynamic intensity modulation method based on orthogonal double-layer grating rotation sweep according to claim 1, wherein the step 3 comprises the following:
A1) determine the initial position of the leaf, the active leaves are at the edge of the field and the passive leaves are at the junction of the quadrants;
A2) solve the leaf motion trajectory, take the optimized field intensity map of the treatment planning system as the optimization objective, use the multi-segment segmented linear function to fit the local surface, carry out the optimization solution to make the intensity map of the orthogonal leaf motion trajectory meet the requirements, and obtain the ray fluence function f₁(x,y) of the active leaves in each quadrant, the ray shielding function g₂(x,y) of the passive leaves in each quadrant, and the monitor unit MU_{Quad} in each quadrant.

7. The dynamic intensity modulation method based on orthogonal double-layer grating rotation sweep according to claim 6, wherein the step 4 comprises the following:
B1) initialize the leaves at quadrant boundary position with serial numbers as Qₓ₁₀, Qₓ₂₀, Q_{y0}, and rank the monitor units in each quadrant from large to small as MUₘₐₓ>MU_{sd}>MUₜₕ>MUₘᵢₙ, if MUₘₐₓ−MUₘᵢₙ<ΔMU, jump out of the subsequent step, wherein ΔMU is the maximum monitor unit difference allowed in the quadrant;
B2) find the quadrant of MUₘᵢₙ and MUₘₐₓ;
if MUₘₐₓ and MUₘᵢₙ respectively in the first quadrant and the second quadrant, adjust the leaves with serial number Qₓ₁ to reduce MUₘₐₓ and to increase MUₘᵢₙ;
if MUₘₐₓ and MUₘᵢₙ respectively in the third quadrant and the fourth quadrant, adjust the leaves with serial number Qₓ₂ to reduce MUₘₐₓ and to increase MUₘᵢₙ;
if MUₘₐₓ and MUₘᵢₙ respectively in the first quadrant and the fourth quadrant, or MUₘₐₓ and MUₘᵢₙ, respectively, in the second quadrant and the third quadrant, then adjust the leaves with serial number Q_{y} to reduce MUₘₐₓ and to increase MUₘᵢₙ;
if MUₘₐₓ and MUₘᵢₙ on the quadrant of the diagonal respectively, and MU_{sd} with MUₘᵢₙ is located in the same line, then adjust the leaves with serial number Q_{y} to reduce MUₘₐₓ and to increase MUₘᵢₙ;
if MUₘₐₓ and MUₘᵢₙ on the quadrant of the diagonal respectively, and MU_{sd} with MUₘᵢₙ, that are in the same column, adjust the leaves with serial number Qₓ₁ and the leaves with serial number Qₓ₂ to reduce MUₘₐₓ and to increase MUₘᵢₙ;
B3) adjust the leaves with serial numbers Qₓ₁, Qₓ₂ and Q_{y}, and perform quadrant segmentation calculation again through step 3 to obtain the ray fluence function f₁(x,y) of active leaves in each quadrant, the ray shielding function g₂(x,y) of passive leaves in each quadrant and the monitor unit MU_{Quad} in each quadrant, and return to step B1.

8. The dynamic intensity modulation method based on orthogonal double-layer grating rotation sweep according to claim 7, where in the step 5 comprises the following:
record the ray fluence function f₁(x,y) of the active leaves, the ray shielding function g₂(x,y) of the passive leaves, and the maximum monitor unit MUₘₐₓ of each quadrant calculated by the last orthogonal segmentation, the ray fluence function f₁(x,y) and the ray shielding function g₂(x,y) are the motion trajectories of active and passive leaves by unit conversion, and MUₘₐₓ is the overall monitor unit MU.

9. Dynamic intensity modulation device based on orthogonal double-layer grating rotation sweep, comprising: a computer and a program implemented with the computer for performing the dynamic intensity modulation method based on orthogonal double-layer grating rotation sweep according to claim 1.
